(19) United States
(12) Patent Application Publication
(10) Pub. No.: US 2010/0297038 A1
(43) Pub. Date: Nov. 25, 2010

(54) Benzimidazole Derivatives And Their Use As Cooling Agents

(75) Inventors: Stefan Michael Furrer, Cincinnati, OH (US); Thomas Scott McCluskey, Amelia, OH (US)

Correspondence Address: CURATOLO SIDOTI CO., LPA, 24500 CENTER RIDGE ROAD, SUITE 280, CLEVELAND, OH, 44145, US

(73) Assignee: Givaudan S.A., Vernier (CH)

(21) Appl. No.: 12/812,713

(22) PCT Filed: Jan. 16, 2009
(86) PCT No.: PCT/CH2009/000018

§ 371 Date: Jul. 13, 2010

Related U.S. Application Data
(60) Provisional application No. 61/011,476, filed on Jan. 17, 2008.

(51) Int. Cl. C07D 401/02; A61K 8/49; C07D 235/04; C07D 403/02; A61Q 11/00; A23G 4/06; A23L 1/226

(52) U.S. Cl. 424/48; 424/49; 546/199; 548/304.4; 548/306.1; 426/3; 426/537

(57) ABSTRACT

The present invention refers to compounds with cooling properties, the compound are of formula (I),

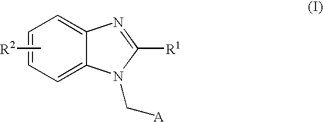

wherein
R¹ is selected from the list consisting of C₁-C₃ alkyl, —SCH₃, and —NH₂, and —CHR′OR″ wherein R′ and R″ are independently selected from hydrogen, methyl and ethyl;
R² is selected from the list consisting of hydrogen, C₁-C₃ alkyl, and a halide; and
I) A is

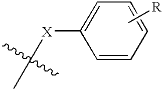

wherein R is selected from hydrogen, —OR¹¹ wherein R¹¹ is selected from hydrogen, and C₁-C₃ alkyl; halide; —NO₂; —CN; —C(O)NH₂; and —C(O)OR¹² wherein R¹² is selected from hydrogen, and C₁-C₃ alkyl; and
X is selected from the list consisting of —CH₂—, —C(O)—, and —C(O)NHCH₂—;
   or

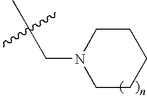

II) A is
   wherein n is 0 or 1.

Provided are a new class of compounds having cooling properties. Also provided are a process of their production and consumer products comprising them.

In the flavour and fragrance industry there is an ongoing demand for compounds having unique cooling properties that provide the user with a pleasing cooling effect and which are suitable for use in a variety of products, particularly in ingestible and topically-applied products.

The most well-known cooling compound is I-menthol, which is found naturally in oil of mint. Since menthol has a strong minty odor and a bitter taste, and provides a burning sensation when used in high concentrations, a variety of other menthyl ester-based and menthyl carboxamide-based cooling compounds have been developed. One that has enjoyed substantial success is N-ethyl p-menthane-carboxamide (WS-3) and is thus also often used as a benchmark.

We have now found a novel class of compounds, which is capable of imparting and/or enhancing a physiological cooling effect in a product in which it is incorporated.

Thus there is provided in a first aspect, the use as cooling agent of a compound of formula (I), or its salts

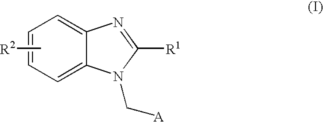

wherein
R¹ is selected from the list consisting of C₁-C₃ alkyl, —SCH₃, and —NH₂, and —CHR′OR″ wherein R′ and R″ are independently selected from hydrogen, methyl and ethyl;
R² is selected from the list consisting of hydrogen, C₁-C₃ alkyl (e.g. ethyl, isopropyl, n-propyl), and halide (e.g. chloride, bromide); and
I) A is

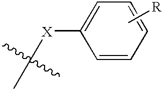

wherein R is selected from hydrogen, —OR¹¹ wherein R¹¹ is selected from hydrogen, and C₁-C₃ alkyl (e.g. methyl); halide (e.g. chloride, bromide); —NO₂; —CN, —C(O)NH₂; and —C(O)OR¹² wherein R¹² is selected from hydrogen, and C₁-C₃ alkyl (e.g. ethyl); and
X is selected from the list consisting of —CH₂—, —C(O)—, and —C(O)NHCH₂—;
   or
II) A is

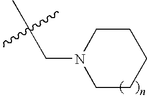

wherein n is 0 or 1.

As used in relation to compounds of formula (I) unless otherwise indicated the term “salts” refers to the salts of the anions chloride, bromide, sulphate, or acetate.

Non limiting examples are compounds of formula (I) wherein R¹ is methyl and A is benzyl which is optionally substituted with methoxy, preferably in para position.

Further none limited examples are compounds of formula (I) wherein R² is hydrogen and A is benzyl which is optionally substituted with methoxy, preferably in para position.

Further none limited examples are compounds of formula (I) wherein R¹ is methyl, R² is hydrogen and A is benzyl which is optionally substituted.

In particular, embodiments are compounds of formula (I) selected from
2-methyl-1-phenethyl-1H-benzo[d]imidazole;
1-(4-methoxyphenethyl)-2-methyl-1H-benzo[d]imidazole;
N-benzyl-2-(2-methyl-1H-benzo[d]imidazol-1-yl)acetamide;
methyl [1-(2-phenylethyl)-1H-benzo[d]imidazol-2-yl]methyl ether;
1-[1-(2-phenylethyl)-1H-benzo[d]imidazol-2-yl]ethanol;
1-[2-(1-pyrrolidinyl)ethyl]-1H-benzo[d]imidazole-2-amine;
2-methyl-1-[2-(1-piperidinyl)ethyl]-1H-benzo[d]imidazole;
1-[2-(1-piperidinyl)ethyl]-1H-benzo[d]imidazole-2-amine;
2-isopropyl-1-phenethyl-1H-benzo[d]imidazole;
1-phenethyl-2-propyl-1H-benzo[d]imidazole; and
1-(1-phenethyl-1H-benzo[d]imidazol-2-yl)propan-1-ol.

The compounds of formula (I) may be used in products that are applied to mucous membranes such as oral mucosa, or to the skin, to give a cooling sensation. By “applying” is meant any form of bringing into contact, for example, oral ingestion, topical application or, in the case of tobacco products, inhalation. In the case of application to the skin, it may be, for example, by including the compound in a cream or salve, or in a sprayable composition. There is therefore also provided a method of providing a cooling sensation to the mucous membrane or skin by applying thereto a product comprising an effective amount of a compound as hereinabove described, or mixtures thereof.

Products that are applied to the oral mucosa may include foodstuffs and beverages taken into the mouth and swallowed, and products taken for reasons other than their nutritional value, e.g. tablets, troches, mouthwash, throat sprays, dentifrices and chewing gums, which may be applied to the oral mucosa for the purpose of cleaning, freshening, healing, and/or deodorizing.

Products that are applied to the skin may be selected from perfumes, toiletries, cosmetic products such as lotions, oils, ointments and bathing agents, applicable to the skin of the human body, whether for medical or other reasons. Accordingly, in a further aspect there is provided a composition comprising an amount of at least one compound of formula (I) sufficient to stimulate the cold receptors in the areas of the skin or mucous membrane with which the composition comes into contact and thereby promote the desired cooling effect. A cooling effect may be achieved upon application of a product, for example, mouthwash or chewing gum, to the mucous membrane, e.g. oral mucosa, comprising less than 5000 ppm, in certain embodiments between 50 and 1000 ppm, such as about 200 ppm, of a compound of formula (I), or mixture thereof. If used for beverages the addition of about 5 ppm may be sufficient to achieve a cooling effect. For use in cosmetic products, the product may comprise from about 50 to about 5000 ppm. However, it is understood that the skilled person may employ compounds of formula (I), as hereinabove described, or a mixture thereof in amounts outside the aforementioned ranges to achieve sensorial effects.

Particular examples of foodstuffs and beverages may include, but are not limited to, beverages, alcoholic or non-alcoholic such as fruit juice beverages, fruit liquors, milk drinks, carbonated beverages, refreshing beverages, and health and nutrient drinks; frozen confectionery such as ice creams and sorbets; desserts such as jelly and pudding; confectionery such as cakes, cookies, chocolates, and chewing gum; jams; candies; breads; tea beverages such as green tea, black tea, chamomile tea, mulberry leaf tea, Roobos tea, peppermint tea; soaps; seasonings; instant beverages; snack foods and the like.

Further examples of products for topical application may include, but are not limited to, skin-care cosmetics such as cleansing tissues, talcum powders, face creams, lotions, tonics and gels; hand creams, hand—and body lotions, anticellulite/slimming creams and—lotions, lotions, balms, gels, sprays and creams; sunburn cosmetics including sunscreen lotions, balms, gels, sprays and creams; after sun lotions, sprays and creams; soaps, toothpicks, lip sticks, agents for bathing, deodorants and antiperspirants, face washing creams, massage creams, and the like,

Further examples of products that are applied to the oral mucosa may include, but are not limited to, oral care products such as toothpastes, tooth gels, tooth powders, tooth whitening products, dental floss, anti-plaque and anti-gingivitis compositions, compositions for treatment of nasal symptoms, and gargle compositions.

Thus there is further provided an end-product selected from the group consisting of products that are applied to the oral mucosa and products that are applied to the skin, such as products for topical application, oral care products, nasal care products, toilet articles, ingestible products and chewing gum, and the like which comprises a product base and an effective amount of at least one cooling compound of formula (I) as defined herein above.

The compounds as hereinabove described may be used alone or in combination with other cooling compounds known in the art, e.g. menthol, menthone, isopulegol, N-ethyl p-menthanecarboxamide (WS-3), N,2,3-trimethyl-2-isopropylbutanamide (WS-23), ethyl 2-(2-isopropyl-5-methylcyclohexanecarboxamido)-acetate (WS-5), menthyl lactate, menthone glycerine acetal (Frescolat® MGA), mono-menthyl succinate (Physcool®), mono-menthyl glutarate, O-menthyl glycerine (CoolAct® 10) and 2-sec-butylcyclohexanone (Freskomenthe®), menthane, camphor, pulegol, cineol, mint oil, peppermint oil, spearmint oil, eucalyptus oil, 3-l-menthoxypropane-1,2-diol, 3-l-menthoxy-2-methylpropane-1,2-diol, p-menthane-3,8-diol, 2-l-menthoxyethane-1-ol, 3-l-menthoxypropane-1-ol, 4-l-menthoxybutane-1-ol, and menthyl pyrrolidone carboxylic acid compounds sold under the commercial name “Questice”. Further examples of cooling compounds can be found e.g. in WO 2005/049553 (e.g. 2-isopropyl-5-methyl-cyclohexanecarboxylic acid (4-cyanomethyl-phenyl)-amide and 2-isopropyl-5-methyl-cyclohexanecarboxylic acid (4-cyano-phenyl)-amide), WO2006/125334 (e.g. 4-((2-isopropyl-5-methyl-cyclohexanecarbonyl)-amino[-benzamide, 3-[(2-isopropyl-5-methyl-cyclohexanecarbonyl)-amino]benzamide, and (2-isopropyl-5-methyl-N-(4-(4-methylpiperazine-1-carbonyl)phenyl)cyclohexanecarboxamide) and WO 2007/019719 (e.g. 2-isopropyl-5-methyl-cyclohexanecarboxylic acid pyridin-2-ylamide, and 2-isopropyl-5-methyl-cyclohexanecarboxylic acid (2-pyridin-2-yl-ethyl)-amide), which are incorporated herein by reference.

Thus there is provided in a further aspect, a composition for cooling comprising a compound of formula (I) as hereinabove defined, or a mixture thereof, optionally combined with at least one other cooling compound.

The cooling compounds of formula (I) may also be blended with known natural sensate compounds, for example, jambu, galangal, galangal acetate, sanshool, capscacian, pepper and ginger, or other flavour and fragrance ingredients generally known to the person skilled in the art. Suitable examples of flavour and fragrance ingredients include alcohols, aldehydes, ketones, esters, ethers, acetates, nitriles, terpene hydrocarbons, nitrogenous, sulphurous heterocyclic compounds, and natural oils, e.g. citrus oil. Flavor and fragrance ingredients may be of natural or synthetic origin. Many of these are listed in reference texts such as the book by S. Arctander, Perfume and Flavor Chemicals, 1969, Montclair, N.J., USA, or its more recent versions.

The cooling compounds may be employed in the products simply by directly mixing the compound with the product, or they may, in an earlier step, be entrapped with an entrapment material such as polymers, capsules, microcapsules and nanocapsules, liposomes, film formers, absorbents such as cyclic oligosaccharides, or they may be chemically bonded to a substrate, which are adapted to release the cooling compound upon application of an external stimulus such as temperature, moisture, and/or enzyme to or the like, and then mixed with the product. Or they may be added while being solubilized, dispersed, or diluted using alcohols or polyhydric alcohols, such as, glycerine, propylene glycol, triazethine and mygliol, natural gums such as gum Arabic, or surfactants, such as glycerine fatty acid esters and saccharide fatty acid esters.

The compounds of formula (I) may either be prepared by alkylation of the appropriate benzimidazole, using a base (e.g. sodium hydride, potassium carbonate, potassium tert, butoxide, sodium hydroxide or potassium hydroxide) and the corresponding alkyl halide; or they may be prepared through a tandem reduction-condensation of a 2-nitro-alkylaniline under condition known to the person skilled in the art, as described, for example, in DE 1021850.

The compositions and methods are now further described with reference to the following non-limiting examples.

These examples are for the purpose of illustration only and it is understood that variations and modifications can be made by one skilled in the art without departing from the scope of the invention. It should be understood that the embodiments described are not only in the alternative, but can be combined.

### EXAMPLE 1

### 2-methyl-1-phenethyl-1H-benzo[d]imidazole

In a round bottom flask 15.0 g of N-methyl benzimidazole were dissolved in N,N-dimethyl formamide (120 mL). 3.36 g of sodium hydride (1.8 eq.) were added to the reaction over the course of 1 hour. The reaction was stirred for 30 min at room temperature, followed by the addition of 14.30 g of 2-bromoethyl benzene (1 eq.) in N,N-dimethyl formamide (30 mL). The reaction was heated at 50° C. for 4 hours. The reaction mixture was diluted with water and extracted using MTBE. The organic layer were washed four times with water, dried over MgSO₄ and concentrated. The crude product was purified using flash chromatography and 2.21 g of 2-methyl-1-phenethyl-H1-benzo[d]imidazole were obtained as a white solid

¹H NMR (DMSO) δ: 7.45-7.36 (m, 2H), 7.26-7.18 (m, 3H), 7.17-7.09 (m, 2H), 7.07-7.01 (d, 2H), 4.40-4.32 (t, 2H), 3.07-3.00 (t, 2H), 2.14 (s, 3H).

¹³C NMR (CDCl₃) δ: 137, 128.84, 128.8, 127.05, 121.97, 121.85, 119.20, 112.2, 109.3, 45.6, 35.81, 13.43.

GC-MS: 236 (M), 145, 118, 104, 92, 77, 65, 51, 39.

### EXAMPLE 2

### 1-(4-methoxyphenethyl)-2-methyl-1H-benzo[d]imidazole

In a 100 mL round bottom flask, 4.24 g of 2-Fluoronitrobenzene, 5.43 g of Diisopropylethylamine (1.4 eq.) and 30 mL of Dimethylsulfoxide were added. 5.0 g of 4-Methoxy-phenethylamine (1.1 eq.) were added and the mixture was heated at 80° C. for 16 h. The mixture was poured under stirring onto 500 mL of ice and the orange solid was filtered off, washed with water and dried under vacuum, to yield 9.3 g of N-(4-methoxyphenethyl)-2-nitroaniline as a yellow solid.

8.0 g of the N-(4-methoxyphenethyl)-2-nitroaniline dissolved in 100 mL of acetic acid and 0.2 g of palladium on charcoal (10%) were added and the mixture was stirred under hydrogen atmosphere for 16 h. The mixture was filtered over celite and the filtrate heated at reflux for 4 h. The reaction mixture was concentrated to about 113 of the volume, diluted with MTBE and NaOH (1N) and extracted. The organic layer was washed with water and brine, dried over MgSO₄, concentrated and purified by column chromatography yield 4.8 g of 1-(4-methoxyphenethyl)-2-methyl-1H-benzo[d]imidazole as a brownish oil, that crystallizes.

¹H NMR (DMSO) δ: 771-7.68 (m, 1H), 7.32-7.22 (m, 3H), 6.87-6.83 (m, 2H), 6.80-6.76 (m, 2H), 4.27 (t, 2H), 3.76 (t, 3H), 3.01 (t, 2H), 2.17 (s, 3H).

¹³C NMR (CDCl₃) δ: 158, 151, 142, 134, 129.79, 129.73, 121.95, 121.82, 119, 109, 55, 46, 35, 13

GC-MS:

### EXAMPLE 3

### N-benzyl-2-(2-methyl-1H-benzo[d]imidazol-1-yl)acetamide

¹H NMR (DMSO) δ: 8.81-8.62 (s, 1H), 7.52-7.45 (m, 1H), 7.4-7.2 (m, 6), 7.18-7.09 (m, 2H), 4.9-4.83 (t, 2H), 4.35-4.28 (m, 2H), 2.5 (s, 3H).

### EXAMPLE 4

### methyl [1-(2-phenylethyl)-1H-benzo[d]imidazol-2-yl]methyl ether

¹H NMR (CDCl₃) δ: 7.80-7.70 (m, 1H), 7.40-7.36 (m, 1H), 7.34-7.20 (m, 5H), 7.11-7.00 (m, 2H), 4.57-4.41 (t, 2H), 4.39 (s, 2H), 3.36 (s, 3H), 3.19-3.05 (m, 2H).

### EXAMPLE 5

### 1-[1-(2-phenylethyl)-1H-benzo[d]imidazol-2-ylethanol

¹H NMR (DMSO) δ: 7.61-7.52 (d, 1H), 7.41-7.35 (d, 1H), 7.33-7.22 (m, 2H), 7.21-7.07 (m, 5H), 5.42-5.31 (d, 1H), 4.81-4.69 (t, 1H), 4.62-4.42 (m, 2H), 3.17-3.06 (t, 2H), 1.60-1.50 (d, 3H).

### EXAMPLE 6

### 1-[2-(1-pyrrolidinyl)ethyl]-1H-benzo[d]imidazole-2-amine

¹H NMR (DMSO) δ: 7.12-7.10 (d, 2H), 6.99-6.78 (m, 2H), 6.39 (s, 2H), 4.13-4.04 (t, 2H), 2.72-2.63 (t, 2H), 2.57-2.44 (m, 4H), 1.75-1.60 (m, 4H).

### EXAMPLE 7

### 2-methyl-1,2-(1-piperidinyl)ethyl]-1H-benzo[d]imidazole

¹H NMR (DMSO) δ: 8.21-8.10 (m, 1H), 7.82-7.73 (m, 1H), 7.62-7.5 (m, 2H), 5.07-4.90 (t, 2H), 3.78-3.38 (m, 2H), 2.91 (s, 3H), 2.50 (s, 4H), 1.85 (s, 6H).

### EXAMPLE 8

### 1-[2-(1-piperidinyl)ethyl]-1H-benzo[d]imidazole-2-amine

¹H NMR (DMSO) δ: 7.17-7.09 (d, 2H), 6.97-6.83 (m, 2H), 6.32 (s, 2H), 4.10-4.00 (t, 2H), 2.60-2.48 (m, 2H), 2.47-2.40 (t, 4H), 1.58-1.31 (m, 6H).

### EXAMPLE 9

### Cooling Intensity

A small group of panelists was asked to taste various aqueous solutions of compounds of formula (I) and indicate which solutions had a cooling intensity similar to or slightly higher than that of a solution of menthol at 2 ppm. The results are shown in Table 1.

TABLE 1

| Chemical | Concentraton | Odor |
|---|---|---|
| Comparison: | 2.0 ppm | Minty |
| I-Menthol | | |
| Comparison: | 1.5 ppm | None |
| N-ethyl p-menthanecarboxamide (WS-3) | | |
| 2-methyl-1-phenethyl-1H-benzo[d]imidazole | 0.03 ppm | None |
| (Example 1) | | |
| 1-(4-methoxyphenethyl)-2-methyl-1H- | 0.1 ppm | None |
| benzo[d]imidazole (Example 2) | | |

### EXAMPLE 10

### Application in Toothpaste

| | composition: | |
|---|---|---|
| Ingredients | A | B |
| Opaque toothgel | 99.20 g | 99.20 g |
| Peppermint oil, terpeneless | 0.50 g | 0.40 g |
| 2-methyl-1-phenethyl-1H-benzo[d]imidazole | 0.00 g | 0.10 g |
| (Example 1) as a 10% solution in | | |
| Peppermint oil, terpeneless | | |
| Saccharin | 0.30 g | 0.30 g |

The materials were mixed in the toothgel, a piece of toothgel was put on a toothbrush and a panelist's teeth were brushed. The mouth was rinsed with water and the water spat out. An intense cooling sensation was felt by the panelist in all areas of the mouth. Whereas the cooling perception lasted for about 40 minutes for composition A (Control), the cooling perception lasted for over 60 minutes for composition B.

### EXAMPLE 11

### Application in Chewing Gum

| | composition: | |
|---|---|---|
| Ingredients | A | B |
| Gum Base Solsona-T | 30 g | 30 g |
| Sorbitol powdered | 50.6 g | 50.6 g |
| Maltitol Syrup 85% | 9 g | 9 g |
| Mannitol powdered | 5 g | 5 g |
| Glycerin | 5 g | 5 g |
| Acesulfame potassium (Ace-K ™) | 0.09 g | 0.09 g |
| Aspartame | 0.21 g | 0.21 g |
| Peppermint oil, terpeneless | 0.50 g | 0.40 g |
| 2-methyl-1-phenethyl-1H-benzo[d]imidazole | 0.00 g | 0.10 g |
| (Example 1) as a 10% solution in Peppermint oil, | | |
| terpeneless | | |

The gum base, and half of the sorbitol were mixed, maltitol syrup was added and then mixed with the gum mass. The rest of the powdered ingredients (rest of the sorbitol, mannitol, ace-K, aspartame) were added and mixed for about 1 minute, at which point glycerine was added and the gum mass was mixed for about 5 minutes, to form the blank chewing gum mass. Peppermint oil (Control; composition A) or the peppermint oil comprising 2-methyl-1-phenethyl-1H-benzo[d]imidazole was worked into the mass and a piece of the resulting gum (2 g) was chewed by a panelist for 20 min and spat out. A cooling sensation was felt by the panelist in all areas of the mouth.

Whereas the cooling perception lasted for about 50 minutes for composition A (Control), the cooling perception lasted for over 60 minutes for composition B.

1. (canceled)

2. A method of providing a cooling sensation to the skin or mucosa membrane by applying thereto a compound of formula (I) 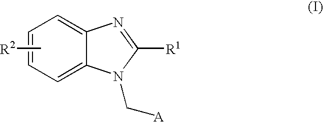
or a salt thereof, wherein
R¹ is selected from the list consisting of C₁-C₃ alkyl; —CHR′OR″ wherein R and R″ are independently selected from hydrogen, methyl and ethyl: —SCH₃: and —NH₂:
R² is selected from the list consisting of hydrogen, C₁-C₃, and a halide; and
I) A is 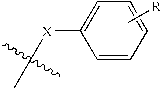
wherein R is selected from hydrogen; —OR¹¹ wherein R¹¹ is selected from hydrogen, and C₁-C₃ alkyl; halide; —NO₂; —CN; —C(O)NH₂; and —C(O)OR¹² wherein R¹² is selected from hydrogen, and C₁-C₃ alkyl: and
X is selected from the list consisting of —CH₂—, —C(O)—, and —C(O)NHCH₂—:
or
II) A is 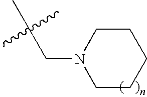
wherein n is 0 or 1.

3. A composition for cooling comprising a compound of formula (I), a salt thereof, or a mixture thereof 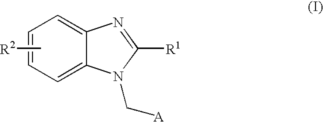
wherein
R¹ is selected from the list consisting of C₁-C₃ alkyl; CHR′OR″ wherein R′ and R″ are independently selected from hydrogen, methyl and ethyl: —SCH₃; and —NH₂;
R² is selected from the list consisting of hydrogen, C₁-C₃, and a halide; and
I) A is 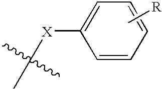
wherein R is selected from hydrogen; —OR¹¹ wherein R¹¹ is selected from hydrogen, and C₁-C₃ alkyl: halide; —CN: —C(O)NH₂; and —C(O)OR¹² wherein R¹² is selected from hydrogen, and C₁-C₃ alkyl: and
X is selected from list consisting of —CH₂—, —C(O)—, and —C(O)NHCH₂—;
or
II) A is 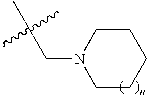
wherein n is 0 or 1.

4. A composition according to claim 3 comprising a compound of formula (I), or a salt thereof, or a mixture thereof and a base material selected from the group consisting of solvents, flavour ingredients and other cooling compounds.

5. A composition according to claim 4 wherein the compound is selected from the list consisting of 2-methyl-1-phenethyl-1H-benzo[d]imidazole;
1-(4-methoxyphenethyl)-2-methyl-1H-1-benzo[d]imidazole;
N-benzyl-2-(2-methyl-1H-benzo[d]imidazol-1-yl)acetamide;
methyl [1-(2-phenylethyl)-1H-benzo[d]imidazol-2-yl]methyl ether;
1-[1-(2-phenylethyl)-1H-benzo[d]imidazol-2-yl]ethanol;
1-[2-(1-pyrrolidinyl)ethyl]-1H-benzimidazol-2-amine;
2-methyl-1-[2-(1-piperidinyl)ethyl]-1H-benzo[d]imidazole;
1-[2-(1-piperidinyl)ethyl]-1H-benzo[d]imidazole-2-amine;
2-isopropyl-1-phenethyl-1H-benzo[d]imidazole;
1-phenethyl-2-propyl-1H-benzo[d]imidazole;
1-(1-phenethyl-1H-benzo[d]imidazol-2-yl)propan-1-ol; and mixtures thereof.

6. A composition according to claim 4 wherein the other cooling compound is selected from the group consisting of menthol, menthone, isopulegol, N-ethyl p-menthanecarboxamide, N,2,3-trimethyl-2-isopropylbutanamide, ethyl-[[5-methyl-2-(isopropyl)cyclohexyl]carbonyl]glycinate, menthyl lactate, menthone glycerine acetal, mono-menthyl succinate, mono-menthyl glutarate, O-menthyl glycerine, 2-sec-butylcyclohexanone, menthane, camphor, pulegol, cineol, mint oil, peppermint oil, spearmint oil, eucalyptus oil, 3-l-menthoxypropane-1,2-diol, 3-l-menthoxy-2-methylpropane-1,2-diol, p-menthane-3,8-diol, 2-l-menthoxyethane-1-ol, 3-l-menthoxypropane-1-ol, 4-l-menthoxybutane-1-ol, 2-isopropyl-5-methyl-cyclohexanecarboxylic acid (4-cyanomethyl-phenyl)-amide, 2-isopropyl-5-methyl-cyclohexanecarboxylic acid (4-cyano-phenyl)-amide, 4-[(2-isopropyl-5-methyl-cyclohexanecarbonyl)-amino]-benzamide, 3-[(2-isopropyl-5-methyl-cyclohexanecarbonyl)-amino]benzamide, (2-isopropyl-5-methyl-N-(4-(4-methylpiperazine-1-carbonyl)phenyl)cyclohexanecarboxamide, 2-isopropyl-5-methyl-cyclohexanecarboxylic acid pyridin-2-ylamide, 2-isopropyl-5-methyl-cyclohexanecarboxylic acid (2-pyridin-2-yl-ethyl)-amide, and mixtures thereof.

7. A composition according to claim 4 wherein the solvent is selected from the group consisting of glycerine, propylene glycol, triazethine and mygliol.

8. A product selected from the group consisting of products that are applied to the oral mucosa and products that are applied to the skin, comprising the composition as defined in claim 3 comprising a product base and an effective amount of a compound of formula (I), a salt thereof, or a mixture thereof.

9. 1-(4-Methoxyphenethyl)-2-methyl-1H-1-benzo[d]imidazole.

10. The composition according to claim 3 wherein the compound is selected From the list consisting of 2-methyl-1-phenethyl-1H-benzo[d]imidazole;
1-(4-methoxyphenethyl)-2-methyl-1H-benzo[d]imidazole;
N-benzyl-2-(2-methyl-1H-benzo[d]imidazol-1-yl)acetamide;
methyl [1-(2-phenylethyl)-1H-benzo[d]imidazol-2-yl]methyl ether;
1-[1-(2-phenylethyl)-1]-1-benzo[d]imidazol-2-yl]ethanol;
1-[2-(1-pyrrolidinyl)ethyl]-1H-benzimidazol-2-amine;
2-methyl-1-[2-(1-piperidinyl)ethyl]-1H-benzo[d]imidazole;
1-[2-(1-piperidinyl)ethyl]-1H-benzo[d]imidazole-2-amine;
2-isopropyl-1-phenethyl-1H-1-benzo[d]imidazole;
1-phenethyl-2-propyl-1H-benzo[d]imidazole;
1-(1-phenethyl-1H-benzo[d]imidazol-2-yl)propan-1-ol; and mixtures thereof.

11. The product according to claim 8 wherein the compound is selected from the list consisting of 2-methyl-1-phenethyl-1H-benzo[d]imidazole;
1-(4-methoxyphenethyl)-2-methyl-1H-1-benzo[d]imidazole;
N-benzyl-2-(2-methyl-1H-benzo[d]imidazol-1-yl)acetamide;
methyl [1-(2-phenylethyl)-1H-benzo[d]imidazol-2-yl]methyl ether;
1-[1-(2-phenylethyl)-1H-benzo[d]imidazol-2-yl]ethanol;
1-[2-(1-pyrrolidinyl)ethyl]-1H-benzimidazol-2-amine;
2-methyl-[2]-(1-piperidinyl)ethyl]-1H-benzo[d]imidazole;
1-[2-(1-piperidinyl)ethyl]-1H-benzo[d]imidazole-2-amine;
2-isopropyl-1-phenethyl-1H-benzo[d]imidazole;
1-phenethyl-2-propyl-1H-benzo[d]imidazole
1-(1-phenethyl-1H-benzo[d]imidazol-2-yl)propan-1-ol; and mixtures thereof.
